(19) United States
(12) Patent Application Publication
(10) Pub. No.: US 2021/0330705 A1
(43) Pub. Date: Oct. 28, 2021

(54) TOLEROGENIC DENDRITIC CELLS FOR TREATMENT OF ACUTE RESPIRATORY DISTRESS SYNDROME

(71) Applicant: Brain Cancer Research Institute, San Diego, CA (US)

(72) Inventors: Thomas Ichim, San Diego, CA (US); Feng Lin, San Diego, CA (US); Sandeep Pingle, San Diego, CA (US); Shashanka Ashili, Rosemount, MN (US)

(21) Appl. No.: 17/237,475

(22) Filed: Apr. 22, 2021

Related U.S. Application Data
(60) Provisional application No. 63/014,282, filed on Apr. 23, 2020.

(51) Int. Cl. A61K 35/15; A61K 31/436

(52) U.S. Cl. CPC A61K 35/15; A61K 31/436

(57) ABSTRACT

Disclosed are compositions of matter, cells, and therapeutic protocols useful for treatment of acute respiratory distress syndrome (ARDS). In some embodiments the invention teaches treatment of ARDS caused by viruses including COVID-19 through administration of an immature population of dendritic cells at a concentration and frequency to inhibit pulmonary inflammation, alveolar leakage, and loss of pulmonary function. In some embodiments immature dendritic cells are generated by culture of autologous and/or allogeneic monocytes with IL-4 and GM-CSF without a maturation step. In other embodiments, generation of immature dendritic cells is performed by administration on NF-kappa B inhibitors. In other embodiments dendritic cells are utilized in an immature form to stimulate generation of T regulatory cells.

### CROSS-REFERENCE TO RELATED APPLICATIONS

This application claims priority to U.S. Provisional Application No. 63/014,282, titled “Tolerogenic Dendritic Cells for Treatment of Acute Respiratory Distress Syndrome”, and filed Apr. 23, 2020, which is hereby incorporated by reference in its entirety.

### FIELD OF THE INVENTION

The invention pertains to the field of treating viral infections through immune modulation, and more specifically the treatment of acute respiratory distress syndromes through the use of dendritic cells.

### BACKGROUND

Acute respiratory distress syndrome (ARDS) is a sudden onset form of respiratory failure caused by a variety of factors. ARDS generally presents with progressive hypoxemia, dyspnea and increased work of breathing [1]. Patients often require mechanical ventilation and supplemental oxygen. Over the years, our understanding of ARDS has advanced significantly. However, ARDS is still associated with significant morbidity and mortality and therapeutic strategies to mitigate the foregoing have resulted in limited translational success. Part of this failure stems from heterogeneity associated with this disease.

ARDS can be caused by bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS. ARDS is often associated with fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Many patients who develop ARDS do not survive. The risk of death increases with age and severity of illness. Of the people who do survive ARDS, some recover completely while others experience lasting damage to their lungs.

While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel therapeutic cells and combinations useful in the treatment of ARDS and methods of treatment for ARDS and conditions related thereto through the administration of such novel therapeutic agents.

### SUMMARY

Various aspects of the invention are directed to methods of treating acute respiratory distress syndrome (ARDS) comprising administering immature dendritic cells, of autologous or allogeneic sources, at a sufficient concentration and frequency sufficient to reduce, ameliorate or reverse ARDS.

Preferred embodiments include methods wherein said ARDS is associated one or more of the following selected from a group consisting of: a) bacterial pneumonia, b) viral pneumonia; c) sepsis; d) head injury; e) chest injury; f) burns; g) blood transfusions; h) near drowning; i) aspiration of gastric contents; j) pancreatitis; k) intravenous drug use; l) abdominal trauma and m) acute radiation syndrome.

Preferred embodiments include methods wherein the administration of immature dendritic cells decreases mRNA levels of inflammatory cytokine(s), increases mRNA levels of anti-inflammatory cytokine(s).

Preferred embodiments include methods wherein the inflammatory cytokine is IL-6, IL1a, TNF-alpha, IL1 beta, Interferon gamma, IL-8, CXCL-1, CCL-2, HMGB-1, IL-11, IL-17, IL-18, IL-21, IL-22, IL-27, IL-33, and TNF-beta.

Preferred embodiments include methods wherein said anti-inflammatory cytokine is selected from a group comprising of: a) IL-10; b) TGF-beta; c) IL-4; d) TGS-6; e) galectin-1; galectin-3; and g) galecin-9.

Preferred embodiments include methods wherein the administration of immature dendritic cells decreases protein levels of an inflammatory cytokine, increases protein levels of an anti-inflammatory cytokine.

Preferred embodiments include methods wherein the inflammatory cytokine is IL-6, IL1a, TNF-alpha, IL1 beta, Interferon gamma, IL-8, CXCL-1, CCL-2, HMGB-1, IL-11, IL-17, IL-18, IL-21, IL-22, IL-27, IL-33, and TNF-beta.

Preferred embodiments include methods wherein said anti-inflammatory cytokine is selected from a group comprising of: a) IL-10; b) TGF-beta; c) IL-4; d) TGS-6; e) galectin-1; galectin-3; and g) galecin-9.

Preferred embodiments include methods wherein said immature dendritic cell is derived from a monocyte precursor.

Preferred embodiments include methods wherein said monocyte precursor is a monocyte.

Preferred embodiments include methods wherein said monocyte precursor is plastic adherent.

Preferred embodiments include methods wherein said monocyte precursor expresses CD14.

Preferred embodiments include methods wherein said monocyte precursor is a type 2 monocyte.

Preferred embodiments include methods wherein said immature dendritic cell is derived from said monocyte precursor by exposing said monocyte precursor to an agent capable activating the GM-C SF receptor.

Preferred embodiments include methods wherein said agent capable of activating said GM-CSF receptor is GM-CSF.

Preferred embodiments include methods wherein monocyte is exposed to said agent capable of activity said GM-CSF receptor for a period of 1 hour to 14 days.

Preferred embodiments include methods wherein monocyte is exposed to said agent capable of activity said GM-CSF receptor for a period of 1 day to 7 days.

Preferred embodiments include methods wherein monocyte is exposed to said agent capable of activity said GM-CSF receptor for a period of 1 day to 2 days.

Preferred embodiments include methods wherein said immature dendritic cell is derived from a hematopoietic stem cell precursor.

Preferred embodiments include methods wherein said hematopoietic stem cell precursor is a hematopoietic stem cell of autologous and/or allogeneic origin.

Preferred embodiments include methods wherein said hematopoietic stem cell is not plastic adherent.

Preferred embodiments include methods wherein said hematopoietic stem cell expresses CD34.

Preferred embodiments include methods wherein said hematopoietic stem cell expresses CD133.

Preferred embodiments include methods wherein said immature dendritic cell is derived from a hematopoietic stem cell by exposing said hematopoietic stem cell to an agent capable activating the GM-CSF receptor.

Preferred embodiments include methods wherein said agent capable of activating said GM-CSF receptor is GM-CSF.

Preferred embodiments include methods wherein hematopoietic stem cell is exposed to said agent capable of activity said GM-CSF receptor for a period of 1 hour to 14 days.

Preferred embodiments include methods wherein hematopoietic stem cell is exposed to said agent capable of activity said GM-CSF receptor for a period of 1 day to 7 days.

Preferred embodiments include methods wherein hematopoietic stem cell is exposed to said agent capable of activity said GM-CSF receptor for a period of 1 day to 2 days.

Preferred embodiments include methods wherein said immature dendritic cell is generated be exposure of a dendritic cell precursor to a combination of GM-CSF and IL-4 for a period of 2-7 days.

Preferred embodiments include methods wherein said immature dendritic cell is generated be exposure of a dendritic cell precursor to a combination of GM-CSF and IL-4 for a period of 3-4 days.

Preferred embodiments include methods wherein said immature dendritic cell expresses CD11c.

Preferred embodiments include methods wherein said immature dendritic cell expresses DEC-205.

Preferred embodiments include methods wherein said immature dendritic cell expresses higher levels of interleukin-10 as compared to its precursor.

Preferred embodiments include methods wherein said immature dendritic cell expresses higher levels of PD-L1 as compared to its precursor.

Preferred embodiments include methods wherein said immature dendritic cell expresses higher levels of TSG-6 as compared to its precursor.

Preferred embodiments include methods wherein said immature dendritic cell is capable of generating T regulatory cells.

Preferred embodiments include methods wherein said T regulatory cells express FoxP3.

Preferred embodiments include methods wherein said T regulatory cells are capable of inhibiting proliferation of T cells that have been stimulating through the T cell receptor and/or a costimulatory molecule.

Preferred embodiments include methods wherein immature dendritic cells are generated in part by culture with an inhibitor of NF-kappa B.

Preferred embodiments include methods wherein said inhibitor of NF-kappa B is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

Preferred embodiments include methods wherein rapamycin is administered in vitro and/or in vivo to suppress dendritic cell maturation.

Preferred embodiments include methods wherein said ARDS is treated by inhibiting cytokine storm in a patient, said inhibition of cytokine storm is accomplished by the steps of: a) obtaining placental tissue; b) dissociating said placental tissue in a manner so as to obtain a single cell suspension; c) extracting from said single cell suspension cells expressing the marker CD14; and d) culturing said cells in GM-CSF and/or IL-4 and GM-CSF at a concentration and frequency sufficient to generate immature dendritic cells.

Preferred embodiments include methods wherein said cytokine storm is excessive production of inflammatory cytokines.

Preferred embodiments include methods wherein said inflammatory cytokines are associated with increasing permeability of blood vessels.

Preferred embodiments include methods wherein said inflammatory cytokines are associated with induction of hypotension.

Preferred embodiments include methods wherein said inflammatory cytokines are associated with induction of vascular leakage.

Preferred embodiments include methods wherein said inflammatory cytokines are associated with an increase in pro-thombotic molecules on the vasculature.

Preferred embodiments include methods wherein said pro-thrombotic molecule on the vasculature is tissue factor.

Preferred embodiments include methods wherein said pro-thrombotic molecule on the vasculature is von Willebrand factor.

Preferred embodiments include methods wherein said pro-thrombotic molecule on the vasculature is plasminogen activator inhibitor.

Preferred embodiments include methods wherein said inflammatory cytokines are associated with reduction of anti-thrombotic factors on endothelial cells.

Preferred embodiments include methods wherein said anti-thrombotic factor on endothelial cells is inducible nitric oxide synthase.

Preferred embodiments include methods wherein said anti-thrombotic factor on endothelial cells is thrombomodulin.

Preferred embodiments include methods wherein said anti-thrombotic factor on endothelial cells is protein C receptor.

Preferred embodiments include methods wherein said inflammatory cytokines are cytokines capable of inducing expression of genes in endothelial cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, homo sapiens Down syndrome critical region, FI5613 containing ZNF gene family member, IL8, ELFR, homo sapiens mRNA for dual specificity phosphatase MKP-5, homo sapiens regulator of G protein signaling 10 mRNA complete, Homo sapiens Wnt-13 Mma, homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 homo sapiens mRNA, CDNA DKFZp434N2072,RPL26, and HR gene for hairless protein, regulator of G protein signaling.

Preferred embodiments include methods wherein said inflammatory cytokines are selected from a group comprising of: a) IL-1; b) IL-6; c) IL-12; d) IL-18; e) IL-33; f) TNF-alpha; g) IFN-gamma; h) HMGB-1; and i) IL-15.

Preferred embodiments include methods wherein said immature dendritic cell therapy is administered together with an immune suppressive agent.

Preferred embodiments include methods wherein said immune suppressive agent inhibits T cell proliferation.

Preferred embodiments include methods wherein said immune suppressive agent inhibits T cell cytokine production.

Preferred embodiments include methods wherein said immune suppressive agent inhibits antigen presenting cell function.

Preferred embodiments include methods wherein said immune suppressive agent inhibits B cell activity.

Preferred embodiments include methods wherein said immune suppressive agent inhibits B cell activity.

Preferred embodiments include methods wherein said immune suppressive agent is selected from a group comprising of: cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), Monoclonal antibodies, basiliximab (Simulect), daclizumab (Zinbryta)

Preferred embodiments include methods wherein a cell therapy is administered in conjunction with immature dendritic cells and/or agents that stimulate immature dendritic cells.

Preferred embodiments include methods wherein said cell therapy is a mesenchymal stem cell.

Preferred embodiments include methods wherein said mesenchymal stem cell expresses markers selected from a group comprising of: a) CD73; b) CD90; c) CD105; d) PD-L1; and e) membrane bound TGF-beta.

Preferred embodiments include methods wherein said mesenchymal stem cell does not express markers selected from a group comprising of: a) HLA II; b) CD14; and c) CD34.

Preferred embodiments include methods wherein said mesenchymal stem cell is plastic adherent.

Preferred embodiments include methods wherein said mesenchymal stem cell possess expression of indolamine 2,3, deoxygenase.

Preferred embodiments include methods wherein said mesenchymal stem cell has been pretreated with an inflammatory stimuli for a time period and concentration sufficient to enhance anti-inflammatory properties of said mesenchymal stem cell.

Preferred embodiments include methods wherein said inflammatory stimuli are selected from a group comprising of: a) interferon gamma; b) interleukin-1; c) interleukin-6; d) interleukin-8; e) TNF-alpha; f) interleukin-11; g) interleukin 12; h) interleukin-15; i) interleukin-17; j) interleukin-18; and k) interleukin-33.

Preferred embodiments include methods wherein enhanced anti-inflammatory activity is increased production of interleukin-10.

Preferred embodiments include methods wherein enhanced anti-inflammatory activity is increased production of TSG-6.

Preferred embodiments include methods wherein enhanced anti-inflammatory activity is increased ability to inhibit production of TNF-alpha from endotoxin activity macrophages.

Preferred embodiments include methods wherein said cell therapy comprises administration of T regulatory cells.

Preferred embodiments include methods wherein said cell therapy comprises administration of type 2 macrophages.

Preferred embodiments include methods wherein said cell therapy comprises administration of myeloid suppressor cells.

Preferred embodiments include methods wherein said cell therapy comprises administration of hematopoietic stem cells.

Preferred embodiments include methods wherein said hematopoietic stem cells express CD34.

Preferred embodiments include methods wherein an agent with direct or indirect anti-viral activity is added for enhancement of therapeutic efficacy.

Preferred embodiments include methods wherein said antiviral agent is chloroquine.

Preferred embodiments include methods wherein said antiviral agent is hydroxychloroquine.

Preferred embodiments include methods wherein said antiviral agent is remdesivir.

Preferred embodiments include methods wherein said antiviral agent is lopinavir.

Preferred embodiments include methods wherein said antiviral agent is Reproxalap.

Preferred embodiments include methods, wherein said antiviral agent is Apabetalone.

Preferred embodiments include methods wherein said antiviral agent is Tradipitant.

Preferred embodiments include methods wherein said antiviral agent is Arbidol umifenovir.

Preferred embodiments include methods wherein said antiviral agent is Ganovo danoprevir.

Preferred embodiments include methods wherein said antiviral agent is Riavax tertomotide.

Preferred embodiments include methods wherein said antiviral agent is Thymosin alpha 1.

Preferred embodiments include methods wherein said antiviral agent is Ifenprodil (NP-120).

Preferred embodiments include methods wherein said antiviral agent is Avigan favipiravir.

Preferred embodiments include methods wherein said antiviral agent is Aviptadil.

Preferred embodiments include methods wherein said antiviral agent is Oseltamivir.

Preferred embodiments include methods wherein immature dendritic cells are maintained in an immature state by gene editing genes associated with dendritic cell maturation.

Preferred embodiments include methods wherein said genes associated with dendritic cell maturation are selected from a group comprising of: a) NF-kappa b; b) interleukin-12; c) CD40; d) CD80; and e) CD86.

Preferred embodiments include methods wherein RNA interference is utilized as a substitute for gene editing in order to maintain dendritic cells in an immature state.

Preferred embodiments include methods wherein antisense oligonucleotides are utilized as a substitute for gene editing in order to maintain dendritic cells in an immature state.

Preferred embodiments include methods wherein ribozymes are utilized as a substitute for gene editing in order to maintain dendritic cells in an immature state.

### DETAILED DESCRIPTION OF THE INVENTION

Disclosed are means of inducing a tolerogenic state in the lung of an individual susceptible to, or, suffering from acute respiratory distress syndrome (ARDS). The invention teaches that administration of immature dendritic cells, of autologous and/or allogeneic origin, provides an environment conducive to stimulation of cells which inhibit inflammation and stimulate regeneration of damaged pulmonary cells. In one embodiment of the invention, patients are identified as having risk of ARDS based on typical clinical parameters and/or cytokine alterations.

The invention, in some embodiments, teaches the application of Immunological tolerance to the condition of ARDS. It is known that a cardinal feature of the immune system, is allowing for recognition and elimination of pathological threats, while selectively ignoring antigens that belong to the body. Traditionally, autoimmune conditions or conditions associated with cytokine storm, such as ARDS are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. The same holds true for cytokine storm conditions such as sepsis, where overproduction of agents such as TNF-alpha result in vascular leakage, coagulopathy, and death. The invention provides the utilization of tolerance-induction in ARDS alone, or in combination with existing techniques. The utilization of antigen-nonspecific immature dendritic cells in ARDS allows for induction of an inhibitory immune response, which results in suppression of pulmonary inflammation.

To “cure” conditions of immune overactivation in ARDS, the invention teaches that it is important to delete/inactivate the T cell clone that are associated with stimulation of inflammation, as well as to block innate immune elements. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [2]; the “mirror image”, T regulatory (Treg) cells are actually selected to live by encounter with self-antigens, and Treg cells that do not bind self antigens are deleted [3, 4]. In one embodiment of the invention, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [5-11].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [12], antigen presentation in the context of PD1-L [13], and HLA-G interacting with immune inhibitory receptors such as ILT4 [14]. In pregnancy, “tolerogenic antigen presentation” occurs only through the indirect pathway of antigen presentation [15]. Other pathways of selective tolerogenesis in pregnancy include the stimulation of Treg cells, which have been demonstrated essential for successful pregnancy [16]. In the context of cancer, depletion of tumor specific T cells, while sparing of T cells with specificities to other antigens has been demonstrated by the tumor itself or tumor associated cells [17-20]. Additionally, Treg cells have been demonstrated to actively suppress anti-tumor T cells, perhaps as a “back up” mechanism of tumor immune evasion [21-23]. At a clinical level the ability of tumors to inhibit peripheral T cell activity has been associated in numerous studies with poor prognosis [24-26]. Oral tolerance is the process by which ingested antigens induce generation of antigen-specific TGF-beta producing cells (called “Th3” by some) [27-29], as well as Treg cells [30, 31]. Ingestion of antigen, including the autoantigen collagen II [32], has been shown to induce inhibition of both T and B cell responses in a specific manner [33, 34]. It appears that induction of regulatory cells, as well as deletion/anergy of effector cells is associated with antigen presentation in a tolerogenic manner [35]. Remission of disease in animal models of RA [36], multiple sclerosis [37], and type I diabetes [38], has been reported by oral administration of autoantigens. Furthermore, clinical trials have shown signals of efficacy of oral tolerance in autoimmune diseases such as rheumatoid arthritis [39], autoimmune uveitis [40], and multiple sclerosis [41]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments of the invention the generation of immature dendritic cells is performed by either coculture in vitro, or administration in vivo of T regulatory cells [42].

In some embodiments of the invention, alpha 1 antitrypsin is administered in order to induce tolerogenic dendritic cells in order to treat ARDS. The use of this compound for stimulation of immature DC has been previously described and is incorporated by reference [43].

In one embodiment immature dendritic cells are administered to treat capillary leak syndrome and/or ARDS. Identification of these two conditions can be made based on techniques which are known in the art, and the methods described herein can be used to reduce, inhibit or alleviate at least one symptom of the disease. Symptoms of capillary leak syndrome (SCLS) include, but are not limited to, for example, low blood pressure (hypotension), hypoalbuminemia, decrease in plasma volume (hemoconcentration), fatigue, nausea, abdominal pain, extreme thirst, increase in body weight, elevated white blood count, fluid accumulation in lower limbs, watery stool, among others. Symptoms of ARDS include, but are not limited to, for example, shortness of breath, cough, fever, fast heart rates, rapid breathing, chest pain, decreased oxygen levels, and pathological symptoms, including, for example, severe alveolar congestion, presence of hemorrhage, interstitial edema and increased alveolar wall thickness, among others.

In some embodiments of the invention, administration of immature dendritic cells is performed using other agents. Some agents include Inhaled nitric oxide (iNO), which is a vasodilator indicated for treatment of term and near-term neonates with hypoxic respiratory failure associated with clinical or echocardiographic evidence of pulmonary hypertension. In these patients, iNO has been shown to improve oxygenation and reduce the need for extracorporeal membrane oxygenation therapy. NO binds to and activates cytosolic guanylate cyclase, thereby increasing intracellular levels of cyclic guanosine 3′,5′-monophosphate (cGMP). This, in turn, relaxes vascular smooth muscle, leading to vasodilatation. Inhaled NO selectively dilates the pulmonary vasculature, with minimal systemic vasculature effect as a result of efficient hemoglobin scavenging. In acute lung injury (ALI) and acute respiratory distress syndrome (ARDS), increases in partial pressure of arterial oxygen (PaO.sub.2) are believed to occur secondary to pulmonary vessel dilation in better-ventilated lung regions. As a result, pulmonary blood flow is redistributed away from lung regions with low ventilation/perfusion ratios toward regions with normal ratios. Unfortunately iNO works in few patients, we therefore, within the scope of the current invention, seek to increase the efficacy of iNO through administration of immature dendritic cells, or exosomes thereof

In one embodiment the invention teaches reduction of Inflammatory cytokines, especially tumor necrosis factor alpha (TNF) and interleukin 1-beta (IL-1), by administration of immature dendritic cells. It is known that these inflammatory cytokines are major mediators that can elicit changes in cell phenotype, especially causing a variety of morphological and gene expression changes in endothelial cells. With respect to coagulation, one of the clot-promoting and one of the inhibitory pathways seem especially prone to modulation by these cytokines. In one embodiment, administration of immature dendritic cells is performed in order to reduce potential for coagulopathy.

It is known that whenever Tissue Factor contacts the blood, coagulation is initiated rapidly, in one embodiment, the immature dendritic cells reduce tissue factor expression by endothelial cells, or cytokines that produce this effect. These cytokines, TNF and IL-1, can elicit Tissue Factor production on endothelium and monocytes. Therefore, in one embodiment of the invention, administration of immature dendritic cells is disclosed in order to induce a profound systemic reduction of IL-1 and TNF at a concentration of modulation sufficient to prevent disseminated intravascular coagulation. In the normal physiological situation Tissue Factor is located exclusively in the extravascular space, largely on fibroblasts, where it is expressed constitutively. Furthermore, cytokines, especially interleukin 6 (IL-6), can stimulate new platelet formation, and the new platelets responding to IL-6 have increased sensitivity to thrombin activation and increased procoagulant activity. Regulating the clotting process are a large number of anticoagulant and fibrinolytic mechanisms. The three major anticoagulant mechanisms appear to involve antithrombin-heparin, Tissue Factor pathway inhibitor (TFPI) and the Protein C pathway. Of these, the Protein C pathway appears to be the primary target for cytokine action. The Protein C pathway is initiated when thrombin binds to thrombomodulin (TM). In one embodiment of the invention ppMSC are utilized to induce upregulation of anti-coagulative proteins. TM is expressed constitutively on endothelium. In tissue culture, TNF, IL-1 or endotoxin lead to a slow loss of TM and endothelial cell Protein C receptor (EPCR) from the cell surface. In addition, Protein S levels decrease in patients with disseminated intravascular coagulation (DIC). Taken together, these results suggest that cytokines should elicit massive thrombotic responses when administered systemically. At near toxic levels, TNF fails to elicit an overt DIC or thrombotic response in patients, although sensitive markers of coagulation do detect changes in coagulation in response to TNF. In one embodiment of the invention, concentrations of TNF and IL-1, as well as pro-coagulant pathway components and anti-coagulant components are used to guide concentration of immature dendritic cell administration. In baboons, very high levels of TNF also fail to elicit fibrinogen or platelet consumption. However, if the Protein C pathway is blocked, these cytokines can elicit either DIC or deep-vein thrombosis, depending on the conditions. Thrombus formation is potently potentiated by impeding flow and/or by catheterization. DIC is facilitated by providing membrane surfaces, possibly mimicking complement mediated platelet activation/damage that occurs in shock [44]. In one embodiment of the invention, microvesicles such as exosomes produced by immature dendritic cells are used to modulate the thrombogenicity of the blood vessel surface to inhibit DIC.

In one embodiment of the invention, immature dendritic cells are utilized to allow for augmentation of endothelial anti-thrombotic functions after a patient receives paclitaxel. In one specific embodiment paclitaxel is given to a ARDS patient and immature dendritic cells are administered to reduce potential thrombosis. In another embodiment, immature dendritic cells for patients with COVID-19. Studies have shown that tissue factor pathway inhibitor expression was reduced by prolonged treatment with either paclitaxel or TNF-alpha [45]. In one embodiment of the invention, immature dendritic cells are administered to increase expression of tissue factor pathway inhibitor expression.

In one aspect of the invention, immature dendritic cells are utilized as biological regulator of inflammation. Under normal conditions, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g. asthma, psoriasis, arthritis, rheumatoid arthritis, multiple sclerosis, inflammatory bowel disease and the like), septic shock and multiple organ failure. Importantly, these diverse disease states share common inflammatory mediators, such as cytokines, chemokines, inflammatory cells and other mediators secreted by these cells. In the context of the current invention immature dendritic cells are utilized to inhibit pathological inflammation while allow various aspects of the immune response to remain intact.

Generally, inflammatory conditions, infection-associated conditions or immune-mediated inflammatory disorders that may be prevented or treated by administration of the immature dendritic cells. Examples of such inflammatory conditions include sepsis-associated conditions, inflammatory bowel diseases, autoimmune disorders, inflammatory disorders and infection-associated conditions. It is also thought that cancers, cardiovascular and metabolic conditions, neurologic and fibrotic conditions can be prevented or treated by administration of the TLR3 antibody antagonists of the invention. Inflammation may affect a tissue or be systemic. Exemplary affected tissues are the respiratory tract, lung, the gastrointestinal tract, small intestine, large intestine, colon, rectum, the cardiovascular system, cardiac tissue, blood vessels, joint, bone and synovial tissue, cartilage, epithelium, endothelium, hepatic or adipose tissue. Exemplary systemic inflammatory conditions are cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, severe viral infections, influenza, pneumonia, shock, or sepsis.

One example of an inflammatory condition that is treatable with immature dendritic cells is sepsis-associated condition that may include systemic inflammatory response syndrome (SIRS), septic shock or multiple organ dysfunction syndrome (MODS). dsRNA released by viral, bacterial, fungal, or parasitic infection and by necrotic cells can contribute to the onset of sepsis. While not wishing to be bound by an particular theory, it is believed that treatment with immature dendritic cells can provide a therapeutic benefit by extending survival times in patients suffering from sepsis-associated inflammatory conditions or prevent a local inflammatory event (e.g., in the lung) from spreading to become a systemic condition, by potentiating innate antimicrobial activity, by demonstrating synergistic activity when combined with antimicrobial agents, by minimizing the local inflammatory state contributing to the pathology, or any combination of the foregoing. Such intervention may be sufficient to permit additional treatment (e.g., treatment of underlying infection or reduction of cytokine levels) necessary to ensure patient survival. Sepsis can be modeled in animals, such as mice, by the administration of D-galactosamine and poly(I:C). In such models, D-galactosamine is a hepatotoxin which functions as a sepsis sensitizer and poly(I:C) is a sepsis-inducing molecule that mimics dsRNA and activates TLR3. immature dendritic cells treatment may increase animal survival rates in a murine model of sepsis, and thus ppMSC may be useful in the treatment of sepsis.

Treatment of gastrointestinal inflammation by immature dendritic cells is also another embodiment of the invention. Specifically, gastric is inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Mucosal layer may be mucosa of the bowel (including the small intestine and large intestine), rectum, stomach (gastric) lining, or oral cavity. Exemplary chronic gastrointestinal inflammatory conditions are inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), infections colitis, ischemic colitis, collagenous or lymphocytic colitis, necrotizing enterocolitis, colitis in conditions such as chronic granulomatous disease or celiac disease, food allergies, gastritis, infectious gastritis or enterocolitis (e.g., Helicobacter pylori-infected chronic active gastritis) and other forms of gastrointestinal inflammation caused by an infectious agent. Inflammatory bowel disease (IBD) includes a group of chronic inflammatory disorders of generally unknown etiology, e.g., ulcerative colitis (UC) and Crohn's disease (CD). Clinical and experimental evidence suggest that the pathogenesis of IBD is multifactorial involving susceptibility genes and environmental factors. In inflammatory bowel disease, the tissue damage results from an inappropriate or exaggerated immune response to antigens of the gut microflora. Several animal models for inflammatory bowel diseases exist. Some of the most widely used models are the 2,4,6-trinitrobenesulfonic acid/ethanol (TNBS)-induced colitis model or the oxazalone model, which induce chronic inflammation and ulceration in the colon. Another model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. Another model involves the adoptive transfer of naive CD45RB.sup.high CD4 T cells to RAG or SCID mice. In this model, donor naive T cells attack the recipient gut causing chronic bowel inflammation and symptoms similar to human inflammatory bowel diseases. The administration of immature dendritic cells of the present invention in any of these models can be used to evaluate the potential efficacy of those antagonists to ameliorate symptoms and alter the course of diseases associated with inflammation in the gut, such as inflammatory bowel disease. Several treatment options for IBD are available, for example anti-TNF-.alpha. antibody therapies have been used for a decade to treat Crohn's disease. However, a significant percentage of patients are refractory to the current treatments, and thus immature dendritic cells appear promising in the treatment of these conditions. In addition, in one aspect of the invention the use of immature dendritic cells together with anti-TNF alpha antibodies are envisions.

In one embodiment of the invention immature dendritic cells are utilized to treat COVID-19 induced, or other types of induced Systemic Inflammatory Response Syndrome (SIRS). According to the accepted definition, this is a term characterizing an inflammatory syndrome caused by infectious or traumatic causes in which patients exhibit at least 2 of the following criteria: 1) Body temperature less than 36° C. or greater than 38° C.; 2) Heart rate greater than 90 beats per minute; 3)Tachypnea, with greater than 20 breaths per minute; or, an arterial partial pressure of carbon dioxide less than 4.3 kPa (32 mmHg: 4) White blood cell count less than 4000 cells/mm³ (4×109 cells/L) or greater than 12,000 cells/mm³ (12×109 cells/L); or the presence of greater than 10% immature neutrophils (band forms) [46]. SIRS is different than sepsis in that in sepsis an active infection is found [47]. These patients may progress to acute kidney or lung failure, shock, and multiple organ dysfunction syndrome. The term septic shock refers to conditions in which the patient has a systolic blood pressure of less than 90 mmHg despite sufficient fluid resuscitation and administration of vasopressors/inotropes.

It is to be noted that immature dendritic cells are generated with the concept of addressing major issues associated with SIRS. Predominant events in the progression to SIRS and subsequently to multiorgan failure (MOR) include: a) systemic activation of inflammatory responses [48]; b) endothelial activation and initiation of the clotting cascade, associated with consumption of anticoagulants and fibrinolytic factors [49]; c) complement activation [50]; and d) organ failure and death.

These pathological events appear to be related to each other, for example, it is known that complement activation stimulates the pro-coagulant state [51]. In the cancer patient SIRS may be initiated by several factors. Numerous patients receive immune suppressive chemo/radiotherapies that promote opportunistic infections [52, 53]. Additionally, given that approximately 40-70% of patients are cachectic, the low grade inflammation causing the cachexia could augment effects of additional bacterial/injury-induced inflammatory cascades [54]. Finally, tumors themselves, and through interaction with host factors, have been demonstrated to generate systemically-acting inflammatory mediators such as IL-1, IL-6, and TNF-alpha that may predispose to SIRS [55, 56].

Current SIRS treatments are primarily supportive. To date, the only drug to have elicited an effect on SIRS in Phase III double-blind, placebo-controlled trials has been Xigris (activated protein C (APC)) [57], which exerts its effects by activating endothelial cell-protecting mechanisms mediating protection against apoptosis, stimulation of barrier function through the angiopoietin/Tie-2 axis, and by reducing local clotting [58-60]. The basis of approval for Xigris has been questioned by some [61] and, additionally, it is often counter-indicated in oncology-associated sepsis (especially leukemias where bleeding is an issue of great concern). In fact, in the Phase III trials of Xigris, hematopoietic transplant patients were excluded [62]. Thus there is a great need for progress in the area of SIRS treatment and adjuvant approaches for agents such as Xigris. In one embodiment of the invention, APC is administered as Xigris.

One of the main causes of death related to SIRS is dysfunction of the microcirculatory system, which in the most advanced stages is manifested as disseminated intravascular coagulation (DIC) [49]. In one embodiment, immature dendritic cells are utilized to inhibit onset of DIC. Without being bound to theory, immature dendritic cells are generated in a manner to inhibit inflammatory mediators associated with SIRS, whether endotoxin or injury-related signals such as TLR agonists or HMGB-1, are all capable of activating endothelium systemically [63, 64]. Under physiological conditions, the endothelial response to such mediators is local and provides a useful mechanism for sequestering an infection and allowing immune attack. In SIRS, the fact that the response is systemic causes disastrous consequences including organ failure. The characteristics of this endothelial response include: a) upregulation of tissue factor (TF) [65, 66] and suppression of endothelial inhibitors of coagulation such as protein C and the antithrombin system causing a pro-coagulant state [67]; b) increased expression of adhesion molecules which elicit, in turn, neutrophil extravasation [68]; c) decreased fibrinolytic capacity [69-71]; and d) increased vascular permeability/non-responsiveness to vaso-dilators and vasoconstrictors [72, 73]. Excellent detailed reviews of molecular signals associated with SIRS-induced endothelial dysfunction have been published[74-82] and one of the key factors implicated has been NF-kB [83]. Nuclear translocation of NF-kB is associated with endothelial upregulation of pro-thrombotic molecules and suppressed fibrinolysis [84-86]. In an elegant study, Song et al. inhibited NF-kB selectively in the endothelium by creation of transgenic mice transgenic expressing exogenous i-kappa B (the NF-kB inhibitor) specifically in the vasculature. In contrast to wild-type animals, the endothelial cells of these transgenic mice experienced substantially reduced expression of tissue factor while retaining expression of endothelial protein C receptor and thrombomodulin subsequent to endotoxin challenge. Furthermore, expression of NF-B was associated with generation of TNF-alpha as a result of TACE activity [87].

It is interesting that the beneficial effects of Xigris in SIRS appear to be associated with its ability to prevent the endothelial dysfunction [88] associated with suppression of proinflammatory chemokines [89], prevention of endothelial cell apoptosis [90], and increased endothelial fibrinolytic activity [91, 92]. Some of the protective activities of Xigris have been ascribed to its ability to suppress NF-kB activation in endothelial cells [93, 94]. Another example of conditions that immature dendritic cells are useful for treatment of is an inflammatory pulmonary condition. Exemplary inflammatory pulmonary conditions include infection-induced pulmonary conditions including those associated with viral, bacterial, fungal, parasite or prion infections; allergen-induced pulmonary conditions; pollutant-induced pulmonary conditions such as asbestosis, silicosis, or berylliosis; gastric aspiration-induced pulmonary conditions, immune dysregulation, inflammatory conditions with genetic predisposition such as as cystic fibrosis, and physical trauma-induced pulmonary conditions, such as ventilator injury. These inflammatory conditions also include asthma, emphysema, bronchitis, chronic obstructive pulmonary disease (COPD), sarcoidosis, histiocytosis, lymphangiomyomatosis, acute lung injury, acute respiratory distress syndrome, chronic lung disease, bronchopulmonary dysplasia, community-acquired pneumonia, nosocomial pneumonia, ventilator-associated pneumonia, sepsis, viral pneumonia, influenza infection, parainfluenza infection, rotavirus infection, human metapneumovirus infection, respiratory syncitial virus infection and aspergillus or other fungal infections. Exemplary infection-associated inflammatory diseases may include viral or bacterial pneumonia, including severe pneumonia, cystic fibrosis, bronchitis, airway exacerbations and acute respiratory distress syndrome (ARDS). Such infection-associated conditions may involve multiple infections such as a primary viral infection and a secondary bacterial infection.

Several clinical studies have supported the possibility that ascorbic acid (AA) mediates a beneficial effect on endothelial cells, especially in the context of chronic stress. Accordingly, in one embodiment of the invention immature dendritic cells are utilized together with AA. Heitzer et al. [95] examined acetylcholine-evoked endothelium-dependent vaso-responsiveness in 10 chronic smokers and 10 healthy volunteers. While responsiveness was suppressed in smokers, administration of intra-arterial ascorbate was capable of augmenting reactivity: an augmentation evident only in the smokers. Endothelial stress induced in 17 healthy volunteers by administration of L-methionine led to decreased responsiveness to hyperemic flow and increased homocysteine levels. Oral AA (1 g/day) restored endothelial responsiveness [96]. Restoration of endothelial responsiveness by AA has also been reported in patients with insulin-dependent [97] and independent diabetes [98], as well as chronic hypertension [99]. In these studies AA was administered intraarterially or intravenously, and the authors proposed the mechanism of action to be increased nitric oxide (NO) as a result of AA protecting it from degradation by reactive oxygen species (ROS).

A closer look at the literature suggests that there are several general mechanisms by which AA may exert endothelial protective properties. The importance of basal production of NO in endothelial function comes from its role as a vasodilator, and an inhibitor of platelet aggregation [100, 101]. High concentrations of NO are pathological in SIRS due to induction of vascular leakage [102]. However, lack of NO is also pathological because it causes loss of microvascular circulation and endothelial responsiveness [103, 104]. Although there are exceptions, the general concept is that inducible nitric oxide synthase (iNOS) and neuronal nitric oxide synthase (nNOS) are associated with sepsis-induced pathologies, whereas eNOS is associated with protective benefits [105]. It is important to note that, while iNOS expression occurs in almost all major cells of the body in the context of inflammation, eNOS is constitutively expressed by the endothelium. AA administration decreases iNOS in the context of inflammation [106, 107], but appears to increase eNOS [108]. Thus, AA appears to increase local NO concentrations through: a) prevention of ROS-mediated NO inactivation [109, 110]; b) increased activity of endothelial-specific nitric oxide synthase (eNOS) [111], possibly mediated by augmenting bioavailability of tetrahydrobiopterin [112-117], a co-factor of eNOS [118]; and c) induction of NO release from plasma-bound S-nitrosothiols [108].

In addition to deregulation of NO, numerous other endothelial changes occur during SIRS, including endothelial cell apoptosis, upregulation of adhesion molecules, and the procoagulant state [119]. AA has been reported to be active in modulating each of these factors. Rossig et al. reported that in vitro administration of AA led to reduction of TNF-alpha induced endothelial cell apoptosis [109]. The effect was mediated in part through suppression of the mitochondria-initiated apoptotic pathway as evidenced by reduced caspase-9 activation and cytochrome c release. To extend their study into the clinical realm, the investigators prospectively randomized 34 patients with NYHA class III and IV heart failure to receive AA or placebo treatment. AA treatment (2.5 g administered intravenously and 3 days of 4 g per day oral AA) Resulted in reduction in circulating apoptotic endothelial cells in the treated but not placebo control group [120]. Various mechanisms for inhibition of endothelial cell apoptosis by AA have been proposed including upregulation of the anti-apoptotic protein bcl-2 [121] and the Rb protein, suppression of p53 [122], and increasing numbers of newly formed endothelial progenitor cells [123].

AA has been demonstrated to reduce endothelial cell expression of the adhesion molecule ICAM-1 in response to TNF-alpha in vitro in human umbilical vein endothelial (HUVEC) cells (HUVEC) [124]. By reducing adhesion molecule expression, AA suppresses systemic neutrophil extravasation during sepsis, especially in the lung [125]. Other endothelial effects of AA include suppression of tissue factor upregulation in response to inflammatory stimuli [126], and effect expected to prevent the hypercoaguable state. Furthermore, ascorbate supplementation has been directly implicated in suppressing endothelial permeability in the face of inflammatory stimuli [127-129], which would hypothetically reduce vascular leakage. Given the importance of NF-kappa B signaling in coordinating endothelial inflammatory changes [84-86], it is important to note that AA at pharmacologically attainable concentrations has been demonstrated to specifically inhibit this transcription factor on endothelial cells [130]. Mechanistically, several pathways of inhibition have been identified including reduction of i-kappa B phosphorylation and subsequent degradation [131], and suppression of activation of the upstream p38 MAPK pathway [132]. In vivo data in support of eventual use in humans has been reported showing that administration of 1 g per day AA in hypercholesterolemic pigs results in suppression of endothelial NF-kappa B activity, as well as increased eNOS, NO, and endothelial function [133]. In another porcine study, renal stenosis was combined with a high cholesterol diet to mimic renovascular disease. AA administered i.v. resulted in suppression of NF-kappa B activation in the endothelium, an effect associated with improved vascular function [134].

An important factor in reports of clinical studies of AA is the difference in effects seen when different routes of administration are employed. Supplementation with oral AA appears to have rather minor effects, perhaps due to the rate-limiting uptake of transporters found in the gut. Indeed, maximal absorption of AA appears to be achieved with a single 200 mg dose [135]. Higher doses produce gut discomfort and diarrhea because of effects of ascorbate accumulation in the intestinal lumen [136]. This is why some studies use parenteral administration. An example of the superior biological activity of parenteral versus oral was seen in a study administering AA to sedentary men. Parenteral but not oral administration was capable of augmenting endothelial responsiveness as assessed by a flow-mediated dilation assay [137].

In some embodiments of the invention immature dendritic cells are administered together with mesenchymal stem cells. “Mesenchymal stem cell” or “MSC” in some embodiments refers to cells that are (1) adherent to plastic, (2) express CD73, CD90, and CD105 antigens, while being CD14, CD34, CD45, and HLA-DR negative, are of autologous and/or allogeneic origin, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

### References

1. Thompson, B. T., R. C. Chambers, and K. D. Liu, Acute Respiratory Distress Syndrome. N Engl J Med, 2017. 377(6): p. 562-572.

2. Ramsdell, F. and B. J. Fowlkes, Clonal deletion versus clonal anergy: the role of the thymus in inducing self tolerance. Science, 1990. 248(4961): p. 1342-8.

3. Apostolou, I., et al., Origin of regulatory T cells with known specificity for antigen. Nat Immunol, 2002. 3(8): p. 756-63.

4. Aschenbrenner, K., et al., Selection of Foxp3+ regulatory T cells specific for self antigen expressed and presented by Aire+ medullary thymic epithelial cells. Nat Immunol, 2007. 8(4): p. 351-8.

5. Cong, Y., et al., Generation of antigen-specific, Foxp3-expressing CD4+ regulatory T cells by inhibition of APC proteosome function. J Immunol, 2005. 174(5): p. 2787-95.

6. Buckland, M., et al., Aspirin-treated human DCs up-regulate ILT-3 and induce hyporesponsiveness and regulatory activity in responder T cells. Am J Transplant, 2006. 6(9): p. 2046-59.

7. Jin, Y., et al., Induction of auto-reactive regulatory T cells by stimulation with immature autologous dendritic cells. Immunol Invest, 2007. 36(2): p. 213-32.

8. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.

9. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.

10. Zhang, X., et al., Generation of therapeutic dendritic cells and regulatory T cells for preventing allogeneic cardiac graft rejection. Clin Immunol, 2008. 127(3): p. 313-21.

11. Marguti, I., et al., Expansion of CD4+ CD25+ Foxp3+ T cells by bone marrow-derived dendritic cells. Immunology, 2009. 127(1): p. 50-61.

12. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.

13. D'Addio, F., et al., The link between the PDL1 costimulatory pathway and Th17 in fetomaternal tolerance. J Immunol, 2011. 187(9): p. 4530-41.

14. Kuroki, K. and K. Maenaka, Immune modulation of HLA-G dimer in maternal-fetal interface. Eur J Immunol, 2007. 37(7): p. 1727-9.

15. Erlebacher, A., et al., Constraints in antigen presentation severely restrict T cell recognition of the allogeneic fetus. J Clin Invest, 2007. 117(5): p. 1399-411.

16. Chen, T., et al., Self-specific memory regulatory T cells protect embryos at implantation in mice. J Immunol, 2013. 191(5): p. 2273-81.

17. Harimoto, H., et al., Inactivation of tumor-specific CD8(+) CTLs by tumor-infiltrating tolerogenic dendritic cells. Immunol Cell Biol, 2013. 91(9): p. 545-55.

18. Ney, J. T., et al., Autochthonous liver tumors induce systemic T cell tolerance associated with T cell receptor down-modulation. Hepatology, 2009. 49(2): p. 471-81.

19. Cheung, A. F., et al., Regulated expression of a tumor-associated antigen reveals multiple levels of T-cell tolerance in a mouse model of lung cancer. Cancer Res, 2008. 68(22): p. 9459-68.

20. Bai, A., et al., Rapid tolerization of virus-activated tumor-specific CD8+ T cells in prostate tumors of TRAMP mice. Proc Natl Acad Sci U S A, 2008. 105(35): p. 13003-8.

21. Jacobs, J. F., et al., Regulatory T cells in melanoma: the final hurdle towards effective immunotherapy? Lancet Oncol, 2012. 13(1): p. e32-42.

22. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.

23. Donkor, M. K., et al., T cell surveillance of oncogene-induced prostate cancer is impeded by T cell-derived TGF-betal cytokine. Immunity, 2011. 35(1): p. 123-34.

24. Whiteside, T. L., Down-regulation of zeta-chain expression in T cells: a biomarker of prognosis in cancer? Cancer Immunol Immunother, 2004. 53(10): p. 865-78.

25. Whiteside, T. L., Signaling defects in T lymphocytes of patients with malignancy. Cancer Immunol Immunother, 1999. 48(7): p. 346-52.

26. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.

27. Faria, A. M. and H. L. Weiner, Oral tolerance. Immunol Rev, 2005. 206: p. 232-59.

28. Weiner, H. L., Induction and mechanism of action of transforming growth factor-beta-secreting Th3 regulatory cells. Immunol Rev, 2001. 182: p. 207-14.

29. Fukaura, H., et al., Induction of circulating myelin basic protein and proteolipid protein-specific transforming growth factor-betal-secreting Th3 T cells by oral administration of myelin in multiple sclerosis patients. J Clin Invest, 1996. 98(1): p. 70-7.

30. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 e1-9.

31. Yamashita, H., et al., Overcoming food allergy through acquired tolerance conferred by transfer of Tregs in a murine model. Allergy, 2012. 67(2): p. 201-9.

32. Park, K. S., et al., Type II collagen oral tolerance; mechanism and role in collagen-induced arthritis and rheumatoid arthritis. Mod Rheumatol, 2009.

33. Womer, K. L., et al., A pilot study on the immunological effects of oral administration of donor major histocompatibility complex class II peptides in renal transplant recipients. Clin Transplant, 2008. 22(6): p. 754-9.

34. Faria, A. M. and H. L. Weiner, Oral tolerance: therapeutic implications for autoimmune diseases. Clin Dev Immunol, 2006. 13(2-4): p. 143-57.

35. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.

36. Thompson, H. S., et al., Suppression of collagen induced arthritis by oral administration of type II collagen: changes in immune and arthritic responses mediated by active peripheral suppression.

Autoimmunity, 1993. 16(3): p. 189-99.

37. Song, F., et al., The thymus plays a role in oral tolerance induction in experimental autoimmune encephalomyelitis. Ann N Y Acad Sci, 2004. 1029: p. 402-4.

38. Hanninen, A. and L. C. Harrison, Mucosal tolerance to prevent type 1 diabetes: can the outcome be improved in humans? Rev Diabet Stud, 2004. 1(3): p. 113-21.

39. Wei, W., et al., A multicenter, double-blind, randomized, controlled phase III clinical trial of chicken type II collagen in rheumatoid arthritis. Arthritis Res Ther, 2009. 11(6): p. R180.

40. Thurau, S. R., et al., Molecular mimicry as a therapeutic approach for an autoimmune disease: oral treatment of uveitis-patients with an MHC-peptide crossreactive with autoantigen first results. Immunol Lett, 1997. 57(1-3): p. 193-201.

41. Weiner, H. L., et al., Double-blind pilot trial of oral tolerization with myelin antigens in multiple sclerosis. Science, 1993. 259(5099): p. 1321-4.

42. Onishi, Y., et al., Foxp3+ natural regulatory T cells preferentially form aggregates on dendritic cells in vitro and actively inhibit their maturation. Proc Natl Acad Sci U S A, 2008. 105(29): p. 10113-8.

43. Ozeri, E., et al., alpha-1 antitrypsin promotes semimature, IL-10-producing and readily migrating tolerogenic dendritic cells. J Immunol, 2012. 189(1): p. 146-53.

44. Esmon, C. T., Possible involvement of cytokines in diffuse intravascular coagulation and thrombosis. Baillieres Best Pract Res Clin Haematol, 1999. 12(3): p. 343-59.

45. Wood, S. C., X. Tang, and B. Tesfamariam, Paclitaxel potentiates inflammatory cytokine-induced prothrombotic molecules in endothelial cells. J Cardiovasc Pharmacol, 2010. 55(3): p. 276-85.

46. American College of Chest Physicians/Society of Critical Care Medicine Consensus Conference: definitions for sepsis and organ failure and guidelines for the use of innovative therapies in sepsis. Crit Care Med, 1992. 20(6): p. 864-74.

47. http://www.youtube.com/watch?v=p2rEJC7He6g.

48. de Jong, H. K., T. van der Poll, and W. J. Wiersinga, The systemic pro-inflammatory response in sepsis. J Innate Immun. 2(5): p. 422-30.

49. Gando, S., Disseminated intravascular coagulation in trauma patients. Semin Thromb Hemost, 2001. 27(6): p. 585-92.

50. Guo, R. F. and P. A. Ward, C5a, a therapeutic target in sepsis. Recent Pat Antiinfect Drug Discov, 2006. 1(1): p. 57-65.

51. Silasi-Mansat, R., et al., Complement inhibition decreases the procoagulant response and confers organ protection in a baboon model of Escherichia coli sepsis. Blood. 116(6): p. 1002-10.

52. Person, A. K., D. P. Kontoyiannis, and B. D. Alexander, Fungal infections in transplant and oncology patients. Infect Dis Clin North Am. 24(2): p. 439-59.

53. Kiehn, T. E., Bacteremia and fungemia in the immunocompromised patient. Eur J Clin Microbiol Infect Dis, 1989. 8(9): p. 832-7.

54. Tisdale, M. J., Cancer cachexia. Curr Opin Gastroenterol. 26(2): p. 146-51.

55. Gelin, J., et al., Role of endogenous tumor necrosis factor alpha and interleukin 1 for experimental tumor growth and the development of cancer cachexia. Cancer Res, 1991. 51(1): p. 415-21.

56. Cahlin, C., et al., Experimental cancer cachexia: the role of host-derived cytokines interleukin (IL)-6, IL-12, interferon-gamma, and tumor necrosis factor alpha evaluated in gene knockout, tumor-bearing mice on C57 BI background and eicosanoid-dependent cachexia. Cancer Res, 2000. 60(19): p. 5488-93.

57. Ely, E. W., G. R. Bernard, and J. L. Vincent, Activated protein C for severe sepsis. N Engl J Med, 2002. 347(13): p. 1035-6.

58. Dhainaut, J. F., et al., Drotrecogin alfa (activated) (recombinant human activated protein C) reduces host coagulopathy response in patients with severe sepsis. Thromb Haemost, 2003. 90(4): p. 642-53.

59. Minhas, N., et al., Activated protein C utilizes the angiopoietin/Tie2 axis to promote endothelial barrier function. FASEB J. 24(3): p. 873-81.

60. Loubele, S. T., H. M. Spronk, and H. Ten Cate, Activated protein C: a promising drug with multiple effects? Mini Rev Med Chem, 2009. 9(5): p. 620-6.

61. Poole, D., G. Bertolini, and S. Garattini, Errors in the approval process and post-marketing evaluation of drotrecogin alfa (activated) for the treatment of severe sepsis. Lancet Infect Dis, 2009. 9(1): p. 67-72.

62. Pastores, S. M., et al., Septic shock and multiple organ failure after hematopoietic stem cell transplantation: treatment with recombinant human activated protein C. Bone Marrow Transplant, 2002. 30(2): p. 131-4.

63. Cristofaro, P. and S. M. Opal, The Toll-like receptors and their role in septic shock. Expert Opin Ther Targets, 2003. 7(5): p. 603-12.

64. Treutiger, C. J., et al., High mobility group 1 B-box mediates activation of human endothelium. J Intern Med, 2003. 254(4): p. 375-85.

65. Lv, B., et al., High-mobility group box 1 protein induces tissue factor expression in vascular endothelial cells via activation of NF-kappaB and Egr-1. Thromb Haemost, 2009. 102(2): p. 352-9.

66. Wada, H., Y. Wakita, and H. Shiku, Tissue factor expression in endothelial cells in health and disease. Blood Coagul Fibrinolysis, 1995. 6 Suppl 1: p. S26-31.

67. Levi, M., The coagulant response in sepsis and inflammation. Hamostaseologie. 30(1): p. 10-2, 14-6.

68. Munro, J. M., J. S. Pober, and R. S. Cotran, Recruitment of neutrophils in the local endotoxin response: association with de novo endothelial expression of endothelial leukocyte adhesion molecule-1. Lab Invest, 1991. 64(2): p. 295-9.

69. Lippi, G., L. Ippolito, and G. Cervellin, Disseminated intravascular coagulation in burn injury. Semin Thromb Hemost. 36(4): p. 429-36.

70. Lau, C. L., et al., Enhanced fibrinolysis protects against lung ischemia-reperfusion injury. J Thorac Cardiovasc Surg, 2009. 137(5): p. 1241-8.

71. Levi, M., M. Schouten, and T. van der Poll, Sepsis, coagulation, and antithrombin: old lessons and new insights. Semin Thromb Hemost, 2008. 34(8): p. 742-6.

72. Shapiro, N., et al., The association of endothelial cell signaling, severity of illness, and organ dysfunction in sepsis. Crit Care. 14(5): p. R182.

73. Druey, K. M. and P. R. Greipp, Narrative review: the systemic capillary leak syndrome. Ann Intern Med. 153(2): p. 90-8.

74. Dejana, E., F. Orsenigo, and M. G. Lampugnani, The role of adherens junctions and VE-cadherin in the control of vascular permeability. J Cell Sci, 2008. 121(Pt 13): p. 2115-22.

75. Azevedo, L. C., et al., Redox mechanisms of vascular cell dysfunction in sepsis. Endocr Metab Immune Disord Drug Targets, 2006. 6(2): p. 159-64.

76. Okajima, K., Prevention of endothelial cell injury by activated protein C: the molecular mechanism(s) and therapeutic implications. Curr Vasc Pharmacol, 2004. 2(2): p. 125-33.

77. Andersson, U. and K. J. Tracey, HMGB1 in sepsis. Scand J Infect Dis, 2003. 35(9): p. 577-84.

78. Strassheim, D., J. S. Park, and E. Abraham, Sepsis: current concepts in intracellular signaling. Int J Biochem Cell Biol, 2002. 34(12): p. 1527-33.

79. ten Cate, H., et al., Microvascular coagulopathy and disseminated intravascular coagulation. Crit Care Med, 2001. 29(7 Suppl): p. S95-7; discussion S97-8.

80. Hawiger, J., Innate immunity and inflammation: a transcriptional paradigm. Immunol Res, 2001. 23(2-3): p. 99-109.

81. Edgington, T. S., et al., Cellular immune and cytokine pathways resulting in tissue factor expression and relevance to septic shock. Nouv Rev Fr Hematol, 1992. 34 Suppl: p. S15-27.

82. Mukaida, N., et al., Novel insight into molecular mechanism of endotoxin shock: biochemical analysis of LPS receptor signaling in a cell free system targeting NF-kappaB and regulation of cytokine production/action through beta2 integrin in vivo. J Leukoc Biol, 1996. 59(2): p. 145-51.

83. Liu, S. F. and A. B. Malik, NF-kappa B activation as a pathological mechanism of septic shock and inflammation. Am J Physiol Lung Cell Mol Physiol, 2006. 290(4): p. L622-L645.

84. Ulfhammer, E., et al., TNF-alpha mediated suppression of tissue type plasminogen activator expression in vascular endothelial cells is NF-kappaB- and p38 MAPK-dependent. J Thromb Haemost, 2006. 4(8): p. 1781-9.

85. Xu, H., et al., Selective blockade of endothelial NF-kappaB pathway differentially affects systemic inflammation and multiple organ dysfunction and injury in septic mice. J Pathol. 220(4): p. 490-8.

86. Ding, J., et al., A pivotal role of endothelial-specific NF-kappaB signaling in the pathogenesis of septic shock and septic vascular dysfunction. J Immunol, 2009. 183(6): p. 4031-8.

87. Song, D., et al., Activation of endothelial intrinsic NF-{kappa}B pathway impairs protein C anticoagulation mechanism and promotes coagulation in endotoxemic mice. Blood, 2009. 114(12): p. 2521-9.

88. Grinnell, B. W. and D. Joyce, Recombinant human activated protein C: a system modulator of vascular function for treatment of severe sepsis. Crit Care Med, 2001. 29(7 Suppl): p. S53-60; discussion S60-1.

89. Brueckmann, M., et al., Activated protein C inhibits the release of macrophage inflammatory protein-1-alpha from THP-1 cells and from human monocytes. Cytokine, 2004. 26(3): p. 106-13.

90. Cheng, T., et al., Activated protein C blocks p53-mediated apoptosis in ischemic human brain endothelium and is neuroprotective. Nat Med, 2003. 9(3): p. 338-42.

91. van Hinsbergh, V. W., et al., Activated protein C decreases plasminogen activator-inhibitor activity in endothelial cell-conditioned medium. Blood, 1985. 65(2): p. 444-51.

92. Sakata, Y., et al., Activated protein C stimulates the fibrinolytic activity of cultured endothelial cells and decreases antiactivator activity. Proc Natl Acad Sci U S A, 1985. 82(4): p. 1121-5.

93. Joyce, D. E. and B. W. Grinnell, Recombinant human activated protein C attenuates the inflammatory response in endothelium and monocytes by modulating nuclear factor-kappaB. Crit Care Med, 2002. 30(5 Suppl): p. S288-93.

94. Brueckmann, M., et al., Drotrecogin alfa (activated) inhibits NF-kappa B activation and MIP-1-alpha release from isolated mononuclear cells of patients with severe sepsis. Inflamm Res, 2004. 53(10): p. 528-33.

95. Heitzer, T., H. Just, and T. Munzel, Antioxidant vitamin C improves endothelial dysfunction in chronic smokers. Circulation, 1996. 94(1): p. 6-9.

96. Chambers, J. C., et al., Demonstration of rapid onset vascular endothelial dysfunction after hyperhomocysteinemia: an effect reversible with vitamin C therapy. Circulation, 1999. 99(9): p. 1156-60.

97. Timimi, F. K., et al., Vitamin C improves endothelium-dependent vasodilation in patients with insulin-dependent diabetes mellitus. J Am Coll Cardiol, 1998. 31(3): p. 552-7.

98. Ting, H. H., et al., Vitamin C improves endothelium-dependent vasodilation in patients with non-insulin-dependent diabetes mellitus. J Clin Invest, 1996. 97(1): p. 22-8.

99. Solzbach, U., et al., Vitamin C improves endothelial dysfunction of epicardial coronary arteries in hypertensive patients. Circulation, 1997. 96(5): p. 1513-9.

100. Gao, Y., The multiple actions of NO. Pflugers Arch. 459(6): p. 829-39.

101. Jackson, W. F., The endothelium-derived relaxing factor. J Reconstr Microsurg, 1989. 5(3): p. 263-71.

102. De Cruz, S. J., N. J. Kenyon, and C. E. Sandrock, Bench-to-bedside review: the role of nitric oxide in sepsis. Expert Rev Respir Med, 2009. 3(5): p. 511-21.

103. Tyml, K., F. Li, and J. X. Wilson, Septic impairment of capillary blood flow requires nicotinamide adenine dinucleotide phosphate oxidase but not nitric oxide synthase and is rapidly reversed by ascorbate through an endothelial nitric oxide synthase-dependent mechanism. Crit Care Med, 2008. 36(8): p. 2355-62.

104. Naseem, K. M., The role of nitric oxide in cardiovascular diseases. Mol Aspects Med, 2005. 26(1-2): p. 33-65.

105. Parratt, J. R., Nitric oxide in sepsis and endotoxaemia. J Antimicrob Chemother, 1998. 41 Suppl A: p. 31-9.

106. Wu, F., K. Tyml, and J. X. Wilson, Ascorbate inhibits iNOS expression in endotoxin- and IFN gamma-stimulated rat skeletal muscle endothelial cells. FEBS Lett, 2002. 520(1-3): p. 122-6.

107. Wu, F., J. X. Wilson, and K. Tyml, Ascorbate inhibits iNOS expression and preserves vasoconstrictor responsiveness in skeletal muscle of septic mice. Am J Physiol Regul Integr Comp Physiol, 2003. 285(1): p. R50-6.

108. Ulker, S., P. P. McKeown, and U. Bayraktutan, Vitamins reverse endothelial dysfunction through regulation of eNOS and NAD(P)H oxidase activities. Hypertension, 2003. 41(3): p. 534-9.

109. Peluffo, G., et al., Superoxide-mediated inactivation of nitric oxide and peroxynitrite formation by tobacco smoke in vascular endothelium: studies in cultured cells and smokers. Am J Physiol Heart Circ Physiol, 2009. 296(6): p. H1781-92.

110. May, J. M., Z. C. Qu, and X. Li, Ascorbic acid blunts oxidant stress due to menadione in endothelial cells. Arch Biochem Biophys, 2003. 411(1): p. 136-44.

111. Heller, R., et al., L-Ascorbic acid potentiates nitric oxide synthesis in endothelial cells. J Biol Chem, 1999. 274(12): p. 8254-60.

112. Nakai, K., et al., Ascorbate enhances iNOS activity by increasing tetrahydrobiopterin in RAW 264.7 cells. Free Radic Biol Med, 2003. 35(8): p. 929-37.

113. d'Uscio, L. V., et al., Long-term vitamin C treatment increases vascular tetrahydrobiopterin levels and nitric oxide synthase activity. Circ Res, 2003. 92(1): p. 88-95.

114. Toth, M., Z. Kukor, and S. Valent, Chemical stabilization of tetrahydrobiopterin by L-ascorbic acid: contribution to placental endothelial nitric oxide synthase activity. Mol Hum Reprod, 2002. 8(3): p. 271-80.

115. Patel, K. B., et al., Oxidation of tetrahydrobiopterin by biological radicals and scavenging of the trihydrobiopterin radical by ascorbate. Free Radic Biol Med, 2002. 32(3): p. 203-11.

116. Stone, K. J. and B. H. Townsley, The effect of L-ascorbate on catecholamine biosynthesis. Biochem J, 1973. 131(3): p. 611-3.

117. Huang, A., et al., Ascorbic acid enhances endothelial nitric-oxide synthase activity by increasing intracellular tetrahydrobiopterin. J Biol Chem, 2000. 275(23): p. 17399-406.

118. Schmidt, T. S. and N. J. Alp, Mechanisms for the role of tetrahydrobiopterin in endothelial function and vascular disease. Clin Sci (Lond), 2007. 113(2): p. 47-63.

119. Keel, M. and O. Trentz, Pathophysiology of polytrauma. Injury, 2005. 36(6): p. 691-709.

120. Rossig, L., et al., Vitamin C inhibits endothelial cell apoptosis in congestive heart failure. Circulation, 2001. 104(18): p. 2182-7.

121. Haendeler, J., A. M. Zeiher, and S. Dimmeler, Vitamin C and E prevent lipopolysaccharide-induced apoptosis in human endothelial cells by modulation of Bcl-2 and Bax. Eur J Pharmacol, 1996. 317(2-3): p. 407-11.

122. Saeed, R. W., T. Peng, and C. N. Metz, Ascorbic acid blocks the growth inhibitory effect of tumor necrosis factor-alpha on endothelial cells. Exp Biol Med (Maywood), 2003. 228(7): p. 855-65.

123. Fiorito, C., et al., Antioxidants increase number of progenitor endothelial cells through multiple gene expression pathways. Free Radic Res, 2008. 42(8): p. 754-62.

124. Mo, S. J., et al., Modulation of TNF-alpha-induced ICAM-1 expression, NO and H2O2 production by alginate, allicin and ascorbic acid in human endothelial cells. Arch Pharm Res, 2003. 26(3): p. 244-51.

125. Martin, W. J., 2nd, Neutrophils kill pulmonary endothelial cells by a hydrogen-peroxide-dependent pathway. An in vitro model of neutrophil-mediated lung injury. Am Rev Respir Dis, 1984. 130(2): p. 209-13.

126. Chen, Y. H., et al., Anti-inflammatory effects of different drugs/agents with antioxidant property on endothelial expression of adhesion molecules. Cardiovasc Hematol Disord Drug Targets, 2006. 6(4): p. 279-304.

127. Bremond, A., et al., Regulation of HLA class I surface expression requires CD99 and p230/golgin-245 interaction. Blood, 2009. 113(2): p. 347-57.

128. Wilson, J. X., Mechanism of action of vitamin C in sepsis: ascorbate modulates redox signaling in endothelium. Biofactors, 2009. 35(1): p. 5-13.

129. Utoguchi, N., et al., Ascorbic acid stimulates barrier function of cultured endothelial cell monolayer. J Cell Physiol, 1995. 163(2): p. 393-9.

130. Bowie, A. and L. A. O'Neill, Vitamin C inhibits NF kappa 8 activation in endothelial cells. Biochem Soc Trans, 1997. 25(1): p. 1315.

131. Carcamo, J. M., et al., Vitamin C suppresses TNF alpha-induced NF kappa 8 activation by inhibiting I kappa 8 alpha phosphorylation. Biochemistry, 2002. 41(43): p. 12995-3002.

132. Bowie, A. G. and L. A. O'Neill, Vitamin C inhibits NF-kappa 8 activation by TNF via the activation of p38 mitogen-activated protein kinase. J Immunol, 2000. 165(12): p. 7180-8.

133. Rodriguez-Porcel, M., et al., Chronic antioxidant supplementation attenuates nuclear factor-kappa 8 activation and preserves endothelial function in hypercholesterolemic pigs. Cardiovasc Res, 2002. 53(4): p. 1010-8.

134. Chade, A. R., et al., Antioxidant intervention blunts renal injury in experimental renovascular disease. J Am Soc Nephrol, 2004. 15(4): p. 958-66.

135. Levine, M., et al., Vitamin C pharmacokinetics in healthy volunteers: evidence for a recommended dietary allowance. Proc Natl Acad Sci U S A, 1996. 93(8): p. 3704-9.

136. Hathcock, J. N., et al., Vitamins E and C are safe across a broad range of intakes. Am J Clin Nutr, 2005. 81(4): p. 736-45.

137. Eskurza, I., et al., Effect of acute and chronic ascorbic acid on flow-mediated dilatation with sedentary and physically active human ageing. J Physiol, 2004. 556(Pt 1): p. 315-24.

1. A method of treating a patient suffering from acute respiratory distress syndrome (ARDS), comprising: administering immature dendritic cells, of autologous or allogeneic sources, at a sufficient concentration and frequency sufficient to reduce, ameliorate or reverse ARDS.

2. The method of claim 1, wherein said ARDS is associated one or more of the following selected from the group consisting of: a) bacterial pneumonia, b) viral pneumonia; c) sepsis; d) head injury; e) chest injury; f) burns; g) blood transfusions; h) near drowning; i) aspiration of gastric contents; j) pancreatitis; k) intravenous drug use; l) abdominal trauma and m) acute radiation syndrome.

3. The method of claim 1, wherein the administration of immature dendritic cells decreases mRNA levels of inflammatory cytokines.

4. The method of claim 3, wherein the inflammatory cytokine is selected from the group consisting of: IL-6, IL1a, TNF-alpha, IL1 beta, Interferon gamma, IL-8, CXCL-1, CCL-2, HMGB-1, IL-11, IL-17, IL-18, IL-21, IL-22, IL-27, IL-33, and TNF-beta.

5. The method of claim 3, wherein said anti-inflammatory cytokine is selected from the group consisting of: a) IL-10; b) TGF-beta; c) IL-4; d) TGS-6; e) galectin-1; galectin-3; and g) galecin-9.

6. The method of claim 1, wherein the administration of immature dendritic cells decreases protein levels of an inflammatory cytokine.

7. The method of claim 5, wherein the inflammatory cytokine is selected from the group consisting of: IL-6, IL1a, TNF-alpha, IL1 beta, Interferon gamma, IL-8, CXCL-1, CCL-2, HMGB-1, IL-11, IL-17, IL-18, IL-21, IL-22, IL-27, IL-33, and TNF-beta.

8. The method of claim 5, wherein said anti-inflammatory cytokine is selected from the group consisting of: a) IL-10; b) TGF-beta; c) IL-4; d) TGS-6; e) galectin-1; galectin-3; and g) galecin-9.

9. The method of claim 1, wherein said immature dendritic cells are derived from a monocyte precursor.

10. The method of claim 9, wherein said monocyte precursor is a monocyte.

11. The method of claim 9, wherein said monocyte precursor is plastic adherent.

12. The method of claim 9, wherein said monocyte precursor expresses CD14.

13. The method of claim 1, wherein immature dendritic cells are generated in part by culture with an inhibitor of NF-kappa B.

14. The method of claim 13, wherein said inhibitor of NF-kappa B is selected from the group consisting of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2C1), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, and Saikosaponin-d, Saline (low Na+ istonic).

15. The method of claim 1, wherein rapamycin is administered in vitro and/or in vivo to suppress dendritic cell maturation.

16. The method of claim 1, wherein said ARDS is treated by inhibiting cytokine storm in a patient, said inhibition of cytokine storm is accomplished by the steps of: a) obtaining placental tissue; b) dissociating said placental tissue in a manner so as to obtain a single cell suspension; c) extracting from said single cell suspension cells expressing the marker CD14; and d) culturing said cells in GM-CSF and/or IL-4 and GM-CSF at a concentration and frequency sufficient to generate immature dendritic cells.

17. The method of claim 16, wherein said cytokine storm is excessive production of inflammatory cytokines.

18. The method of claim 17, wherein said inflammatory cytokines are associated with increasing permeability of blood vessels.

19. The method of claim 17, wherein said inflammatory cytokines are associated with induction of hypotension or vascular leakage or thrombosis.

20. The method of claim 1, wherein exosomes of dendritic cells are utilized to prevent or treat ARDS.
